(19) United States
(12) Patent Application Publication
(10) Pub. No.: US 2025/0032187 A1
(43) Pub. Date: Jan. 30, 2025

(54) Automatic Orthopedic Surgery Planning Systems and Methods

(71) Applicant: PEEK HEALTH S.A., Braga (PT)

(72) Inventors: João Pedro de Araújo Ribeiro, Braga (PT); Sara Isabel Azevedo Da Silva, Braga (PT); Jaime Frederico Bastos De Campos, Braga (PT)

(21) Appl. No.: 18/713,985

(22) PCT Filed: Nov. 16, 2022
(86) PCT No.: PCT/PT2022/050031

§ 371 Date: May 28, 2024

(30) Foreign Application Priority Data
Nov. 29, 2021 (PT) 117610

(51) Int. Cl. A61B 34/10; A61B 6/50; G16H 20/40; G16H 30/40; G16H 50/50

(52) U.S. Cl. CPC A61B 34/10; G16H 20/40; G16H 30/40; G16H 50/50; A61B 6/505; A61B 2034/102

(57) ABSTRACT

The present application describes an automatic orthopedic surgery planning system and method thereof. Embodiments described herein include a computer-implemented method for automatic orthopedic surgery planning comprising the steps of: importing at least one orthopedic medical image from a patient into a software application; selecting a medical procedure to apply to the imported orthopedic medical image; generating a bone model and landmark position of the imported orthopedic medical image; adjusting the landmark position of the imported orthopedic medical image; automatically create a pre-operative planning proposal for the orthopedic medical image; validation of the proposed automatic pre-operative planning proposal; and data file export of the orthopedic surgery planning proposal in case of positive validation.

### TECHNICAL FIELD

The present application describes an automatic orthopedic surgery planning system and method thereof.

### BACKGROUND

Document WO2016110816A1 describes a solution that intends to solve the problem of providing an orthopedic surgery planning system with a stereoscopic vision of the patient's lesion. Disclosed is an orthopedic surgery planning system, where a conjunction window of a 2D and 3D environments, comprises an image's axial plan (301), an image's coronal plan (302), an image' sagittal plan (303), a 3D model of an anatomical structure (304), a library templates (305), isosurfaces (306), measurements of distances and angles (307), orientation cube (308), and multi plans 2D in the 3D environments (309). The applicability of this technology expands to the various areas such as orthopedics, orthodontics, implantology and veterinary. In the referred areas exists a surgery planning involving the reconstruction of bone structures, which is accompanied by the preparation of the material to implant in the patient. This solution has the deficiency of being limited to execute most of the pre-operative planning manually by user interaction, merely presenting the images and models to the user for user-performed pre-operative planning.

Document EP3470006A1 in a general approach discloses an automated segmentation of three-dimensional images of a bony structure, useful in particular for the field of computer assisted surgery, diagnostics, and surgical planning. The method may be summarized as including: i) receiving, by at least one processor, learning data including a plurality of batches of labeled image sets, each image set including image data representative of a bony structure, and each image set including at least one label which identifies the region (segment) of a particular part of the anatomical structure depicted in each image of the image set; ii) training, by the at least one processor, a fully convolutional neural network (CNN) model to segment at least one part of the anatomical structure utilizing the received learning data; and iii) storing, by the at least one processor, the trained CNN model in the at least one nontransitory processor-readable storage medium of the machine learning system. This solution presents some problems and disadvantages, such as being limited to creating automated segmentation of three-dimensional images of bony structure images, not being able to perform computer assisted surgery, diagnostics, and/or surgical planning, meaning this solution is complementary to surgical processes, but does not perform pre-operative diagnosis or the pre-operative planning, while the invention presented in this document does, in addition to the automatic segmentation and landmark detection and classification. In addition, the system is limited to performing bone segmentation and classification and landmarks detection, not performing bone quality evaluations relevant for the pre-operative planning of orthopedic surgical procedures, such as osteophytes detection and classifications of bone density, assessments that are performed by the solution presented herein, relevant for performing the pre-operative diagnosis. In addition, the solution presented herein further allows the complete pre-operative planning of surgical procedures based on the pre-operative diagnosis, which includes automatic bone alignment based on clinical angles, automatic bone resections, and automatic template dimensioning and planning. Document US 2005/059873 A1 discloses a solution related to a method and an apparatus for the preoperative planning and simulation of orthopedic surgical procedures that use medical imaging. The preoperative planning includes the acquisition, calibration, and medical image registration, as well as the reduction of the fracture or the selection of the prosthesis, the application of fixative elements and the creation of the planning report. The described method is composed of: a) obtaining the medical image; b) segmenting the anatomical structure of the medical image, such as bone, but not limiting itself only to bone segments, and manipulating the image segments to simulate a desirable result of the orthopedic surgical procedure; c) marking segments of anatomical structures in medical images; d) the performance of different measurements and analysis, such as the difference in length, angle measurements, well of as as sets more complex measurements, such as deformity analysis, structural links in terms of distances and angles between each other; e) planning that comprises means for producing output images. This solution presents some problems and disadvantages, such as not using the full potential of CT scan and MRI images; the fact that it only permits the cutoff in three different viewing axes which does not allow a stereoscopic vision; the impossibility of combining the cutting in different axes, precluding a clear and accurate view of what the plan to be performed; and if this is done with an X-ray without marker, this medical image can no longer be used for the planning process of orthopedic surgery. Thus, the solution is dependent on how the imaging study was performed.

However, the previous state of the art technologies resorted to the need of a great amount of user interaction to proceed correctly with the planning system.

### SUMMARY

The present application describes automatic orthopedic surgery planning systems and methods thereof.

Embodiments described herein perform an automated procedure of a plurality of the processes of the system patented previously in the document WO2016110816A1. The disclosure herein presented resulted from improvements made by Artificial Intelligence (AI) and Neural Networks, in particular through Deep Learning which introduced AI models to the system that enable the automatic pre-operative planning of orthopedic surgery. Furthermore, the system is limited to performing bone segmentation and classification and landmarks detection, not performing bone quality evaluations relevant for the pre-operative planning of orthopedic surgical procedures, such as osteophytes detection and classifications of bone density, assessments that are performed by the solution presented herein. Furthermore, the disclosure herein presented includes some embodiments that provide a method for integrating the user preferences in the system for future pre-operative plans, complementing the individual proposed plans with user preferences, which may also be used to improve the AI models that perform the automatic pre-operative plan. Furthermore, some embodiments enable the planning system to be used without the need of installing the software application into a physical local hardware device, since it is able to operate on a remote web version via web browser.

The present disclosure describes a computer-implemented method for automatic orthopedic surgery planning comprising the steps of importing at least one orthopedic medical image from a patient (conventional and/or DICOM images) into a software application; selecting a medical procedure to apply to the imported orthopedic medical image; generating a bone model and surgical landmark position (surgical landmark is a designated orientation mark used as guidepost to lead the surgeon, and will be hereinafter referred to as landmark) of the imported orthopedic medical image; adjusting the landmark position of the imported orthopedic medical image; automatically create a pre-operative planning proposal for the orthopedic medical image; validation of the proposed automatic pre-operative planning proposal; and data file export of the orthopedic surgery planning proposal in case of positive validation.

The herein disclosed method is intended to obtain automatic orthopedic surgery planning, consisting mainly of three workflow methods: the pre-operative workflow, the training workflow, and the user preferences workflow.

In some respects, the disclosure provides a method for performing the automatic pre-operative planning of a procedure, described on this disclosure as a pre-operative workflow. In a proposed embodiment of the present disclosure, at least one conventional and/or DICOM medical image representing an anatomical structure, for example of bones, vessels, skin, and muscles, from a patient is imported into the software application. The medical image can be comprised of a conventional image, for example Portable Network Graphics (*.png), joint photographic group image (*.jpg), tagged image file format (*.tiff), or DICOM images, for example, Digital Radiography (X-ray), Computed Tomography (CT), Magnetic Resonance Imaging (MRI), Positron Emission Tomography (PET) among other image types. The medical image is introduced into the system by the user from a communication and storage system (PACS), a Compact-Disk (CD), a folder, a Universal Serial Bus (USB) device or an external storage device. Further, these medical images are automatically anonymized and collected to train the system's AI Model (upon the user's consent) on the training workflow of the invention.

Yet in another proposed embodiment of the present disclosure, upon user manual selection of the medical procedure, the system selects the most appropriate tools, for example specific materials and measurement tools to be used for each surgical procedure. For example, in case the user is performing a Total Knee Arthroplasty, all measurements displayed will be for that procedure in particular, such as the Anatomical Axis of the Femur, Anatomical Axis of the Tibia, Mechanical Axis of the Lower Limb or resection planes, as well as the specific templates needed to correct the deformity.

Yet in another proposed embodiment of the present disclosure, the system automatically generates a digital representation of the patient's anatomy reconstructed from the image(s) imported, in the form of a bone model that can be displayed in a two-dimensional (2D) or a three-dimensional (3D) environment.

In another proposed embodiment of the present disclosure, the system automatically detects, positions and labels relevant landmarks for the procedure on the digital representation created, based on the automatic pre-operative diagnosis generated by the training workflow of the invention.

In another proposed embodiment of the present disclosure, the bone model generated can be visualized, rotated, zoomed, and interacted by the user and the landmarks position can be adjusted and altered on the user interface display to refine the position of the landmarks.

Some embodiments of the present disclosure further include a method for automatically performing the pre-operative plan of the procedure including the steps of:
automatically performing bone alignment based on clinical angles detected by the landmarks position. The system detects misalignments and suggests the degrees needed for alignment and correction of the patient's deformity.
automatically detecting the need for bone resections based on the misalignments detected by the landmarks position. The system suggests the angle and degree of the cut needed to correct the deformity. Further, the system allows the user to generate fragments which can be identified as anatomical structures to fix misaligned structures.
automatically dimensioning and placement of templates considering the size and anatomical characteristics of the bone. The system predicts and proposes the most suitable size and type of implant from its comprehensive digital template database displayed for each procedure. Further, the template is automatically added to the digital image and positioned based on patient's the real surgical considerations.
adaptation according to the user preferences, based on the information collected by the system on the user's past interactions with the software, disclosed later in the user preferences workflow. Manual alterations made to the pre-operative planning are detected and the information is collected for the system to learn and adapt in accordance with the preferences of the individual user. This information is integrated in the system for future pre-operative plans, complementing the proposed plan with user preferences.

These steps result in an automatic pre-operative planning proposal, which allows user interaction in the user interface, including repositioning, measurement of distances and/or angles, intersecting templates 3D models and anatomical structure 3D models, resection of anatomical structure 3D models, templates 3D models dimensioning or replacement, or zooming in a 3D environment, allowing the manual adjustment and refinement of the automatic pre-operative proposal.

Some embodiments of the present disclosure further allow the software application to enquire the user if the pre-operative planning is to be approved or if manual refinements are needed. In the case the user accepts the pre-operative planning, the software application may export the pre-operative planning report data file. The full report can be downloaded from the system, saved in a suitable document format (e.g., text file, Microsoft™ Word™, Microsoft™ Excel™, Open Document Format, PDF, Hypertext Markup Language (HTML), etc.), and/or locally printed, and/or sent to a PACS. Further, the pre-operative plan can be integrated with external devices or software with Application Programming Interfaces (API), for the purposes of surgical execution of the particular surgical procedure, for example patient-specific instruments, robotics, or navigation systems of augmented reality.

In the case the user does not approve the planning, the pre-operative planning can be adjusted manually, and therefore, the user preference workflow is initiated. In some embodiments, the system presents interactive user interface controls that allow a series of actions on the user interface controls, including a 3D environment area and a 2D environment area with three 2D plans: axial, coronal and sagittal. These two-dimensional environments may be linked, which means, whenever an element is moved in an environment, this change is automatically reflected in the other environment accordingly. In addition to the linking of the two environments, the user can still position the 2D plans on the 3D model of the anatomical structure, to improve the accuracy.

Embodiments of the present disclosure also provide a method for integrating the user preferences in the automatic pre-operative planning of a procedure, described in this disclosure as the user preferences workflow. When the pre-operative planning is adjusted by the user, the system detects and collects the information regarding the user preferences in the procedure. The system collects all data on the changes manually performed, and the software's Artificial Intelligence is trained to use this data to personalize the procedures in accordance with the user's preferences and habits. A statistical model for user preferences is created to learn the personal preferences of individual users to provide tailored suggestions to the automatic pre-operative planning step of the pre-operative workflow. Furthermore, all data collected on the changes manually performed by the user may be sent to be manually reviewed by a professional prepared and/or accredited for the analysis and study of medical images, who may include the manually performed alterations into the AI model training based on annotated datasets when they are considered an improvement to the proposed planning.

Embodiments of the present disclosure also provide a working structure of the software application that results in a set of AI models that are applicable in the pre-operative diagnosis of the patient's problem, described in this disclosure as the training workflow, comprising the steps of acquiring and storing orthopedic medical images; labeling the stored orthopedic medical images in annotated datasets; providing the labeled datasets to an AI model for training; detecting and classifying bones and landmarks in orthopedic medical images through the trained AI models; generating AI models based on the AI training for bones and landmarks detection step; performing the evaluation of the bone quality; performing accuracy testing of the AI Models generated from the previous steps; result analysis of the accuracy testing; training the AI model to detect and classify bones and landmarks and the following steps are repeated in case of a negative analysis; model inclusion in the pre-operative diagnosis of the result analysis in case of a positive analysis; performing a pre-operative diagnosis analysis for further data inclusion on the bone model and landmark position generation.

Yet in another proposed embodiment of the present disclosure, the training workflow includes the steps of medical imaging acquisition and storing, complemented with the reception of anonymized images from the software application, representing anatomical structures, for example of bones, vessels, skin, and muscles, that can be a conventional and/or DICOM image. The medical images are manually labeled for relevant landmarks and bones, from which results in annotated datasets used for training the system's Artificial Intelligence model, i.e., a script provides instructions to read the datasets and train the AI model for the automatic detection and classification of landmarks and bones, based on the annotated ground truth of the dataset.

Yet in another proposed embodiment of the present disclosure, the workflow performs the bone quality evaluation, comprised of a bone density classification model and an osteophytes detection model, described as follows:
Model for the classification of bone density based on the automatic analysis of the anatomical bone to detect its mineral density and adjust the pre-operative diagnosis with this information.
Model for osteophytes detection based on the automatic analysis of the anatomical bone to detect the presence of bone spurs and adjust the pre-operative diagnosis with this information.

Yet in another proposed embodiment of the present disclosure, the accuracy testing comprises the steps of automatic testing and human verification. The results obtained from testing may be evaluated as satisfactory or not satisfactory. Models with satisfactory results will be included in the system to be used for the pre-operative diagnosis, and not satisfactory models will be sent back to the step of AI model training to detect and classify bones and landmarks.

Yet in another proposed embodiment of the present disclosure, the pre-operative diagnosis comprises the steps of automatic segmentation and classification, automatic landmarks detection, automatic classification of bone density and automatic osteophytes detection.

Yet in another proposed embodiment of the present disclosure, the automatic pre-operative diagnosis resulting from the training workflow is integrated into the pre-operative workflow of the invention, creating the generation of the digital representation of the patient's anatomy reconstructed from the image(s) imported into the system.

The present disclosure further describes a data processing system, comprising the physical means necessary for the execution of the computer-implemented method for automatic orthopedic surgery planning.

The herein disclosed system may include the following units:
a three-dimensional template model database;
a medical imaging study of the patient;
a data processing means;
a memory;
a user interface;
a multiplanar rendering module;
a three-dimensional environment module.

The present disclosure further describes a computer program, comprising the programming code or instructions suitable for carrying out the computer-implemented method for automatic orthopedic surgery planning, in which said computer program is stored, and is executed in said data processing system, remote or in-site, for example a server, performing all the actions previously described.

The present disclosure further describes a computer readable physical data storage device, in which the programming code or instructions of the computer program previously described are stored.

The three-dimensional template model database may include a digital template library comprised of different sets of orthopedic implants and their parameters, from several different orthopedic manufacturing companies, and is meant to be used in the automatic prediction and position of the most suitable implant for the procedure in accordance with the patient's particular anatomical features, also considering the template's configurations. In addition, the user has access to the template database and can refine the preoperative planning by choosing another implant (type, brand, size, etc.) in alternative to the proposed implant. Similar Computer-Assisted Orthopedic Surgery (CAOS) systems resort to the use of virtual implant databases storing the templates of several manufacturers, which is also used in the present invention, however, the database used in this innovation is selected in accordance to the user's preferences, and, in addition, the database is remotely controlled, which allows to obtain a safety benefit in the case of a template recall, when the template can be immediately removed from the database, ensuring patient safety.

In some embodiments, the medical imaging study of the patient may include the study of the medical images imported to the system (conventional and/or DICOM images) using AI models for the detection and classification of anatomical bones and landmarks, the analysis of clinical angles to detect misalignments and deformities, the assessment of bone density, and the detection of osteophytes, and is used to accurately generate the bone model, to accurately perform the automatic pre-operative diagnosis of the patient's problem, as well as to inform the pre-operative planning of the procedure.

Typically, the patient imaging study includes the manual or automatic digitization of points in the medical image of a bone to locate anatomical landmarks and bone segmentation and classification to provide intraoperative navigational guidance. In contrast, some embodiments described herein include a method to perform an automatic imaging study through the generation and training of AI models for automatic segmentation and classification, automatic landmarks detection, automatic classification of bone density, and automatic osteophytes detection that allows to obtain the automatic generation of the bone model and landmark position, the automatic pre-operative diagnosis and the automatic pre-operative planning proposal, in which the user's manual interaction is optional to refine the process.

The data processing means are related to the use of an adequate local or remote apparatus, accessed through a communications network, configured to execute all the processing tasks of the herein disclosed method. In some embodiments, the local and/or remote apparatuses may include an internal local or remote memory, or computer-readable medium, as well as a interface/computer program that will allow to communicate and interact all the technical features of the method with the user.

The multiplanar rendering module may include an image generation model configured to receive the information from the medical image imported to the system and the results generated by the pre-operative diagnosis models, using Computer Graphics based technology, used to generate the 3D bone model of the patient's anatomy, displayed in the user's interface, and allows the visualization in 3 different orthogonal planes, Axial, Coronal and Sagittal, of the patient's image. However, when the Coronal and the Sagittal plane are inexistent, the system digitally creates and processes it from the (original) Axial plane. This is displayed to the user via 3 orthogonal planes, crossing each other in the (0,0,0) coordinate plane. Furthermore, the multiplanar rendering model also allows rotation, and interaction with the image.

The bi-dimensional and/or three-dimensional environment module may include the toolbar and the object edition section, where the user is able to visualize and interact with the virtual image created by the system through Augmented Reality technology and is used to allow the user to visualize, zoom, rotate, measure distances and/or angles, intersect template 3D models and anatomical 3D structure models, reposition landmarks and/or templates, and interact, at the same time, with the virtual 2D and 3D environments to be able to analyze and refine the pre-operative plan. Similar environment modules resort to the use of Augmented Reality (AR) to generate three-dimensional environments that allow user visualization and interaction. However, the present invention allows to achieve a greater level of detail from the image, with more viewable structures, due to the stereoscopic vision that facilitates the identification of bone problems, fractures extension, etc.

### BRIEF DESCRIPTION OF THE DRAWINGS

For better understanding of the present application, figures representing preferred embodiments are herein attached which, however, are not intended to limit the technique disclosed herein.

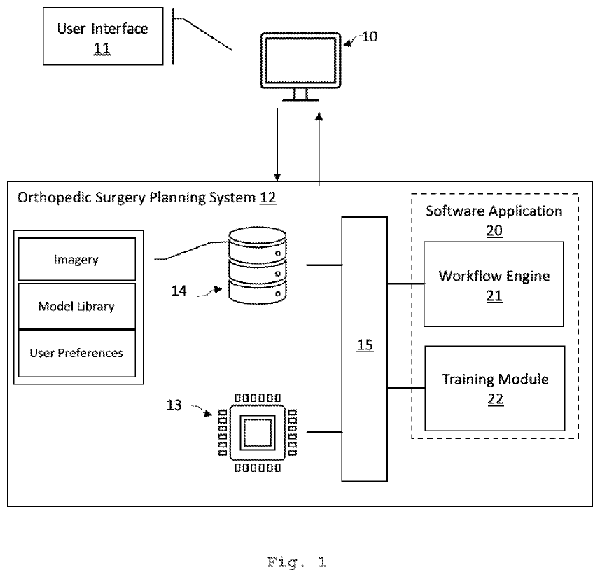

FIG. 1—illustrates an automatic orthopedic surgery planning system in accordance with one or more embodiments of the present disclosure, wherein the reference numbers relate to:
10—User computing device;
11—User interface;
12—Orthopedic surgery planning system;
13—Processor;
14—Storage;
15—Communication bus;
20—Software application;
21—Workflow engine;
22—Training module;

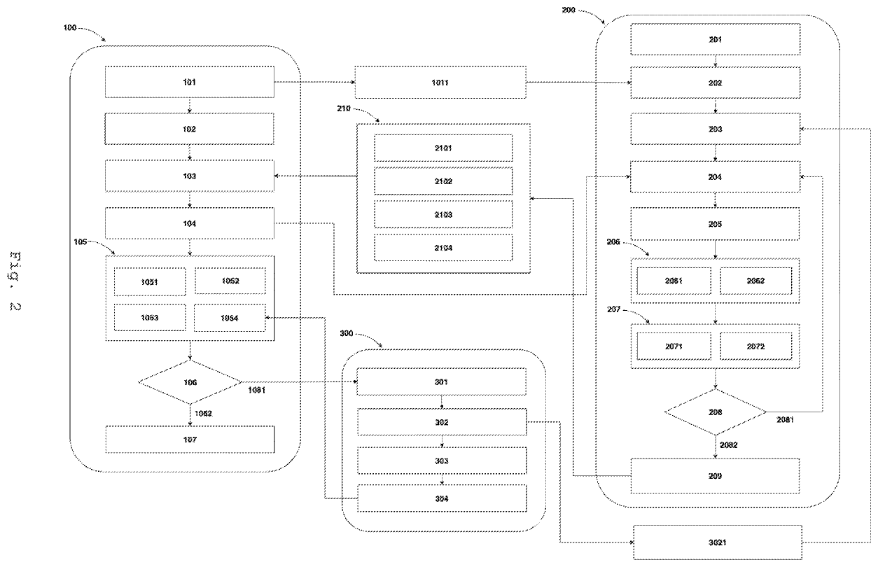

FIG. 2—illustrates the workflow of the disclosed automatic orthopedic surgery planning system in accordance with one or more embodiments of the present disclosure, wherein the reference numbers relate to:
100—Pre-operative workflow;
101—Import image data of the patient;
1011—Receive anonymized images;
102—Select medical procedure;
103—Generate the bone model and landmark position;
104—Landmark position adjustment;
105—Automatic pre-operative planning proposal;
1051—Automatic bone alignment based on clinical angles;
1052—Automatic bone resections;
1053—Automatic template dimensioning and placement;
1054—User preferences;
106—User approves the pre-operative plan?;
1061—No;
1062—Yes;
107—pre-operative planning data file/report export integrable with robotics, PSI and navigation systems;
200—training workflow;
201—Medical Images acquired and stored;
202—Manual labeling;
203—AI model training based on annotated/labeled datasets;
204—AI models training to detect and classify bones and landmarks;
205—AI Models generation;
206—Bone quality evaluation;
2061—Model Classification for bone density;
2062—Model for osteophytes detection;
207—Testing: results are checked and measured in terms of accuracy;
2071—Automatic testing;
2072—Human verification;
208—results satisfactory?;
2081—No;
2082—Yes;
209—Models inclusion in the system;
210—Pre-operative diagnosis;
2101—Automatic segmentation and classification;
2102—Automatic landmarks detection;
2103—Model for automatic classification of bone density;
2104—Model for automatic osteophytes detection;
300—user preferences workflow;
301—User adjusts the pre-operative planning;
302—User preferences settings;
3021—Manual review of user preferences
303—AI training module;
304—Statistical model for user preferences;

### DESCRIPTION OF EMBODIMENTS

With reference to the figures, some embodiments are now described in more detail, which are however not intended to limit the scope of the present application.

Various detailed embodiments of the present disclosure, taken in conjunction with the accompanying figures, are disclosed herein; however, it is to be understood that the disclosed embodiments are merely illustrative. In addition, each of the examples given in connection with the various embodiments of the present disclosure is intended to be illustrative, and not restrictive.

Throughout the specification, the following terms take the meanings explicitly associated herein, unless the context clearly dictates otherwise. The phrases “in one embodiment” and “in some embodiments” as used herein do not necessarily refer to the same embodiment(s), though it may. Furthermore, the phrases “in another embodiment” and “in some other embodiments” as used herein do not necessarily refer to a different embodiment, although it may. Thus, as described below, various embodiments may be readily combined, without departing from the scope or spirit of the present disclosure.

In addition, the term “based on” is not exclusive and allows for being based on additional factors not described, unless the context clearly dictates otherwise. In addition, throughout the specification, the meaning of “a,” “an,” and “the” include plural references. The meaning of “in” includes “in” and “on.”

As used herein, the terms “and” and “or” may be used interchangeably to refer to a set of items in both the conjunctive and disjunctive in order to encompass the full description of combinations and alternatives of the items. By way of example, a set of items may be listed with the disjunctive “or”, or with the conjunction “and.” In either case, the set is to be interpreted as meaning each of the items singularly as alternatives, as well as any combination of the listed items.

FIGS. 1 and 2 illustrate systems and methods of anatomical modelling and simulation. The following embodiments provide technical solutions and technical improvements that overcome technical problems, drawbacks and/or deficiencies in the technical fields involving pre-operative planning using anatomical modelling and simulation. As explained in more detail, below, technical solutions and technical improvements herein include aspects of improved automated medical image processing for operation simulation to enable improved pre-operative planning using specially designed and trained artificial intelligence (AI) and/or machine learning models. Based on such technical features, further technical benefits become available to users and operators of these systems and methods. Moreover, various practical applications of the disclosed technology are also described, which provide further practical benefits to users and operators that are also new and useful improvements in the art.

Embodiments of the present disclosure may include one or more methods to obtain automatic orthopedic surgery planning that may include the following workflow methods: the pre-operative workflow (100), the training workflow (200) and the user preferences workflow (300).

The mentioned workflows, in one of the preferred embodiments, operate under a software application supported by a hardware device properly configured for that purpose.

For example, in some embodiments, the orthopedic surgery planning system (12) may receive the medical images for automated analysis, orthopedic surgery planning, model training, and user preference learning. In some embodiments, the orthopedic surgery planning system (12) may be a part of the user computing device (10). Thus, the orthopedic surgery planning system (12) may include hardware and software components including, e.g., user computing device (10) hardware and software, cloud or server hardware and software, or a combination thereof.

In some embodiments, the orthopedic surgery planning system (12) may include hardware components such as a processor (13), which may include local or remote processing components. In some embodiments, the processor (13) may include any type of data processing capacity, such as a hardware logic circuit, for example an application specific integrated circuit (ASIC) and a programmable logic, or such as a computing device, for example, a microcomputer or microcontroller that include a programmable microprocessor. In some embodiments, the processor (13) may include data-processing capacity provided by the microprocessor. In some embodiments, the microprocessor may include memory, processing, interface resources, controllers, and counters. In some embodiments, the microprocessor may also include one or more programs stored in memory.

Similarly, the orthopedic surgery planning system (12) may include storage (14) for storing imagery (e.g., medical imagery, digital implants database), machine learning and/or statistical models, and/or user preference data. In some embodiments, the storage (14) may include one or more local and/or remote data storage solutions such as, e.g., local hard-drive, solid-state drive, flash drive, database or other local data storage solutions or any combination thereof, and/or remote data storage solutions such as a server, mainframe, database or cloud services, distributed database or other suitable data storage solutions or any combination thereof. In some embodiments, the storage (14) may include, e.g., a suitable non-transient computer readable medium such as, e.g., random access memory (RAM), read only memory (ROM), one or more buffers and/or caches, among other memory devices or any combination thereof.

In some embodiments, the orthopedic surgery planning system (12) may run the software application (20), implement computer engines for implementing workflows. In some embodiments, the terms “computer engine” and “engine” identify at least one software component and/or a combination of at least one software component and at least one hardware component which are designed/programmed/configured to manage/control other software and/or hardware components (such as the libraries, software development kits (SDKs), objects, etc.).

Examples of hardware elements may include processors, microprocessors, circuits, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), logic gates, registers, semiconductor device, chips, microchips, chip sets, and so forth. In some embodiments, the one or more processors may be implemented as a Complex Instruction Set Computer (CISC) or Reduced Instruction Set Computer (RISC) processors; x86 instruction set compatible processors, multi-core, or any other microprocessor or central processing unit (CPU). In various implementations, the one or more processors may be dual-core processor(s), dual-core mobile processor(s), and so forth.

Examples of software may include software components, programs, applications, computer programs, application programs, system programs, machine programs, operating system software, middleware, firmware, software modules, routines, subroutines, functions, methods, procedures, software interfaces, application program interfaces (API), instruction sets, computing code, computer code, code segments, computer code segments, words, values, symbols, or any combination thereof. Determining whether an embodiment is implemented using hardware elements and/or software elements may vary in accordance with any number of factors, such as desired computational rate, power levels, heat tolerances, processing cycle budget, input data rates, output data rates, memory resources, data bus speeds and other design or performance constraints.

In some embodiments, the software application (20) may include a workflow engine (21) for implementing the workflows for the orthopedic surgery planning workflow methods as described below. In some embodiments, the workflow engine (21) may include dedicated and/or shared software components, hardware components, or a combination thereof. For example, the workflow engine (21) may include a dedicated processor and storage. However, in some embodiments, the workflow engine (21) may share hardware resources, including the processor and storage (14) of the orthopedic surgery planning system (12) via, e.g., a bus (15). Thus, the workflow engine (21) may include a memory including software and software instructions, such as, e.g. machine learning models and/or logic.

In some embodiments, the software application (20) may include a training module (22) for training machine learning/Artificial Intelligence and/or statistical models for the orthopedic surgery planning workflow methods as described below. In some embodiments, the training module (22) may include dedicated and/or shared software components, hardware components, or a combination thereof. For example, the training module (22) may include a dedicated processor and storage. However, in some embodiments, the training module (22) may share hardware resources, including the processor (13) and storage (14) of the orthopedic surgery planning system (12) via, e.g., a bus (15). Thus, the training module (22) may include a memory including software and software instructions, such as, e.g. machine learning models and/or logic.

The pre-operative workflow (100) includes importing at least one medical image from a patient (101), selecting the medical procedure (102), generating the bone model and landmark position (103), landmark position adjustment (104), automatic pre-operative planning proposal (105), pre-operative plan user approval (106) and pre-operative planning data file export, integrable with robotics, PSI and navigation systems (107).

Importing an image data of the patient (101) is the first step of the pre-operative workflow (100) wherein the user interacting with an orthopedic surgery planning software application (20) (“software application (20)”) running on an orthopedic surgery planning system may import at least one medical image (conventional and DICOM images). In some embodiments, the user may include a medical specialist operating the software application (20).

In one of the preferred embodiments, the medical image is imported (101) from a picture archiving and communication system (PACS), a Compact-Disk (CD), a folder or a Universal Serial Bus (USB) device or an external storage device. Said images provide representations of an anatomical structure of a patient, and include for example bones, vessels, skin, and muscles. This initial step (101) is performed in a 2D environment or a 3D environment, where the user can notice and select the anatomical structure.

In some embodiments, the medical procedure selection (102) may be performed by the user. The user may specify the medical procedure intended to be performed. In some embodiments, based on the medical procedure, the software application (20) may automatically select the most appropriate tools, including determining and displaying specific materials and/or measurement tools applicable to the selected procedure. In some embodiments, the software application (20) may be configured to determine and display the materials and/or measurement tools for each procedure of multiple possible procedures. For example, in the case the user is performing a Total Knee Arthroplasty, all measurements displayed will be for that particular procedure, like the Anatomical Axis of the Femur, Anatomical Axis of the Tibia, Mechanical Axis of the Lower Limb or resection planes. Then the proper implant is selected according to the patient's anatomy and size, ending with the proper alignment of the lower limb, by simulating the placement of the implants and resections needed accordingly.

The bone model and landmark position generation (103) is performed after selecting the medical procedure (102) and automatically generates a 2D and/or 3D bone model of the patient. The bone model and landmark position generation (103) is conducted by the training workflow (200). The bone model generated by the bone model and landmark position generation (103) is reconstructed from the image imported by the user (101), being generated by the data processing means from the extraction of a polygonal mesh of an isosurface from a three-dimensional scalar field, called voxels. The 2D or 3D reconstruction of the bone model occurs and allows the visualization, zoom, rotation, measurement of distances and/or angles, reposition landmarks, and interaction with the image. Moreover, considering the type of procedure and the unique characteristics of the patient's anatomy, specific surgical landmarks are automatically identified and placed on top of the image.

In some embodiments, the bone model may be presented on the user interface (11) such that the user is able to visualize, zoom, rotate, measurement of distances and/or angles, reposition landmarks, and interact with the obtained images, as well as to confirm if the landmarks detection was performed correctly in the landmark position adjustment (104) step. If the landmark position needs refinement, the user may manually, adjust and correct its positioning to the correct place. This adjustment will inform the system's Artificial Intelligence (AI) model (204), which will, in its turn, refine the landmarks detection capabilities on the training workflow (200), enhancing its own performance in future diagnosis.

In some embodiments, following the landmark position adjustment (104), the software application (20) may perform the automatic pre-operative planning proposal (105). In some embodiments, to form the automatic pre-operative planning proposal (105), the user instructs the system to automatically “start planning”, said procedure lasting only a few seconds. This planning is able to adapt itself to different environments by automatically determining that bidimensional images do not have a third dimension, unlike three dimensional images.

In some embodiments, an automatic pre-operative planning proposal interface environment of the software application (20) for the automatic pre-operative planning proposal (105) may have two common sections: the toolbar and the object edition and can be operated in 2D or hybrid. The pre-operative planning proposal (105) comes with distinct clinical procedures and measurement tools specific for each subspecialty (e.g., hip, knee, spine, upper limb, foot, ankle, and trauma).

Further, the automatic pre-operative planning proposal (105) includes the following modules: automatic bone alignment based on clinical angles (1051), automatic bone resections (1052), automatic template dimensioning and placement (1053) and user preferences (1054).

Alignment refers to how the head, shoulders, spine, hips, knees, and ankles relate and line up with each other. To alleviate pain and restore function, bones can be surgically realigned and fixed. Based on a deep learning technology for bone segmentation, the developed methodology has the ability of accurately and automatically analyzing and assessing the bone alignment based on the pre-operative analysis made on the previous stage, i.e., in the automatic pre-operative planning proposal (105). Based on the position of the landmarks, the automatic bone alignment based on clinical angles module (1051) is configured to detect misalignments and, through machine learning mechanisms, artificial intelligence, and computer vision, it can suggest the angles needed for alignment to correct potential existing bone deformities in the patient.

In some embodiments, the software application (20) may be configured to utilize one or more exemplary AI/machine learning techniques for the automatic pre-operative planning proposal (105), as well in other uses as described below. In some embodiments, the AI/machine learning techniques may include, e.g., decision trees, boosting, support-vector machines, neural networks, nearest neighbor algorithms, Naive Bayes, bagging, random forests, and the like. In some embodiments and, optionally, in combination of any embodiment described above or below, an exemplary neutral network technique may be one of, without limitation, feedforward neural network, radial basis function network, recurrent neural network, convolutional network (e.g., U-net) or other suitable network. In some embodiments and, optionally, in combination of any embodiment described or above below, an exemplary implementation of Neural Network may be executed as follows:
i) define Neural Network architecture/model,
ii) transfer the input data to the exemplary neural network model,
iii) train the exemplary model incrementally,
iv) determine the accuracy for a specific number of timesteps,
v) apply the exemplary trained model to process the newly-received input data,
vi) optionally and in parallel, continue to train the exemplary trained model with a predetermined periodicity.

In some embodiments and, optionally, in combination of any embodiment described above or below, the exemplary trained neural network model may specify a neural network by at least a neural network topology, a series of activation functions, and connection weights. For example, the topology of a neural network may include a configuration of nodes of the neural network and connections between such nodes. In some embodiments and, optionally, in combination of any embodiment described above or below, the exemplary trained neural network model may also be specified to include other parameters, including but not limited to, bias values/functions and/or aggregation functions. For example, an activation function of a node may be a step function, sine function, continuous or piecewise linear function, sigmoid function, hyperbolic tangent function, or other type of mathematical function that represents a threshold at which the node is activated. In some embodiments and, optionally, in combination of any embodiment described above or below, the exemplary aggregation function may be a mathematical function that combines (e.g., sum, product, etc.) input signals to the node. In some embodiments and, optionally, in combination of any embodiment described above or below, an output of the exemplary aggregation function may be used as input to the exemplary activation function. In some embodiments and, optionally, in combination of any embodiment described above or below, the bias may be a constant value or function that may be used by the aggregation function and/or the activation function to make the node more or less likely to be activated.

A bone resection is the removal of a portion or growth of bone. The medical term for this minor surgery is called an osteotomy, or literally, “removal of bone”. The decision to perform bone resections depends upon several measurements, crucial to evaluate the severity of the deformity. The automatic bone resections module (1052) is configured to measure and locate the deformity based on the evaluation performed by the automatic pre-operative analysis proposal (landmarks' misalignment) and suggest the angle and degree of the removal of bone needed to correct the deformity.

Joint replacement is a surgical procedure that entails removing part or the entirety of a damaged joint and installing hardware (implant/template) to allow the limb to move without pain or limitations. With the information provided by the bone model and landmark position (103) and possible landmark position adjustments (104), considering the size and anatomical characteristics of the bone, the automatic template dimensioning and placement (1053) may define the most suitable size and type of implant for the patient's unique anatomy, in the case one is needed for the surgical procedure.

Automatic template dimensioning and placement (1053) has a comprehensive digital template (implants) database, and for each subspecialty, this database is filtered to display only the templates for the chosen procedure. The template is automatically added to the image, and it is placed based on the unique surgical considerations and characteristics of each patient, being able to accurately detect the correct size of implant, as well as its anatomical placement, taking into account the bone density evaluation and osteophytes detection models integrated into the generation of the bone model and landmark position (103) step.

To perform the automatic pre-operative planning proposal (105), the system also considers the information collected from the user's interaction with the software application (20), i.e., user preferences (1054). Whenever the user makes manual modifications to the automatic pre-operative planning proposal (105), the system detects this behavior and learns the preferences this individual user has, and integrates this information to the user future planning, saving the information into the user profile using machine learning technology, complementing the proposed plan with the user preferences (1054).

The automatic pre-operative planning proposal (105) performed by the system (the steps 1051, 1052, 1053 and 1054), suggests a pre-operative planning to solve the patient's problem. The pre-operative planning (105) is presented in the graphical interface of the software application (20), where the user can visualize the plan and interact with it—allowing the user to visualize, zoom, rotate, measure distances and/or angles, intersect template 3D models and anatomical 3D structure models, reposition landmarks and/or templates, and interact with the virtual 3D environment to be able to analyze and refine the pre-operative plan.

Following the automatic pre-operative planning proposal (105), the software application (20) may present an enquiry the user via the user interface (11) if the pre-operative planning is approved (106) or if manual refinements and/or alterations performed by the user are needed. In the case the user accepts the pre-operative planning (1062), the software application (20) may export the pre-operative planning data file report (107). If the user does not approve the planning (1061), the automatic pre-operative planning proposal (105) may be adjusted manually, and as a consequence, the user preference workflow (300) is initiated.

At the end of the pre-operative planning, a pre-operative planning data file report (107) is generated with the information regarding the performed planning. The data file report (107) is then saved in a suitable document format (e.g., text file, Microsoft™ Word™, Microsoft™ Excel™, Open Document Format, PDF, Hypertext Markup Language (HTML), etc., and/or locally printed, and/or sent to a PACS.

The data file report (107) and the information provided in it, can also be integrated with external devices or software for the purposes of surgical execution to aid the surgeon during surgery procedures. Such devices and software can be patient-specific instruments, robotics, and navigation systems of augmented reality, among others. The integration with external devices and/or software may be performed using, e.g., an authenticated Application Programming Interface (API), an intermediary software that allows two applications to communicate with each other.

In some embodiments, the term “application programming interface” or “API” refers to a computing interface that defines interactions between multiple software intermediaries. An “application programming interface” or “API” defines the kinds of calls or requests that can be made, how to make the calls, the data formats that should be used, the conventions to follow, among other requirements and constraints. An “application programming interface” or “API” can be entirely custom, specific to a component, or designed based on an industry-standard to ensure interoperability to enable modular programming through information hiding, allowing users to use the interface independently of the implementation.

The user preferences workflow (300) may be initiated in the background of the software application (20) and is not visible or apparent to the end user. The steps of this workflow may result in the integration of the user's preferences in the system and include user adjustment of the pre-operative planning settings (301), user preferences settings (302), manual review of user preferences (3021), AI training settings (303) and model for user preferences (304).

In some embodiments, if the user operating the software application (20) intends to refine the resulting automatic pre-operative planning proposal (105) and thus does not approve the pre-operative plan (1061), a series of actions on the created 3D models can be performed. These actions are performed through adjustment of the pre-operative planning (301).

The user software application (20) interface provides a 3D environment area and a 2D environment area with three 2D plans: axial, coronal and sagittal. These two-dimensional environments are linked, which means whenever an element is moved in an environment, this change is automatically reflected in the other environment. In addition to the linking of these two environments, the user can still position the 2D plans on the 3D model of the anatomical structure, to improve the accuracy. Thus, the user is able to see exactly the planning in a stereoscopic and realistic perspective of what is expected to be encountered at the time of the surgery. The carefully designed UX/UX allows the user to easily perform modifications and adjustments to the suggested pre-operative planning (301).

Whenever the user performs adjustments to the pre-operative planning proposal (105), the user preferences settings (302) may be responsible for collecting all data on the changes made. The collected data on the alterations and refinements manually performed may be used to optimize the decisioning process and to personalize the procedures according to the user's preferences in future pre-operative planning performed by this user. Thus, the system may be able to predict the user's preferences (302) and add the user's preferences (302) to the next planning.

Based on the information collected on the user preferences (302) step, the adjustments performed by the user undergo a manual review (3021) executed by a professional prepared and/or accredited for the analysis and study of medical images, who may include the manually performed alterations into the AI model training (203) when they are considered an improvement to the automatic pre-operative planning proposal (105) by the professional.

Based on the information collected on user preferences (302), the system's Artificial Intelligence module (303) can personalize the next procedures in accordance with the behavior of the user in the previous pre-operative planning, using Preference Learning, that based on the observed preferences of the user allows the system to learn this information and leads the software to adapt the pre-operative planning to the usual user's intentions. The Artificial Intelligence module (303) may utilize one or more machine learning models of the AI/machine learning techniques as described above.

Subsequently, a statistical model (304) is created to train the software to learn the personal preferences of each individual user. Thus, the statistical model (304) may facilitate training the Artificial Intelligence module (303) to predict tailored suggestions to the pre-operative planning, based on the preferences of each individual user's predicted needs, allows the system to make more accurate predictions in future pre-operative planning.

In some embodiments, the training workflow (200) may include the steps of: medical imaging acquisition and storing (201), manual labeling (202), AI models training based on annotated datasets (203), AI models training to detect and classify bones and landmarks (204), AI models generation (205), bone quality evaluation (206), accuracy testing (207), result analysis (208), Model inclusion in the system (209), and the pre-operative diagnosis (210). The training workflow (200) may be initiated and performed in a working substructure of the software application (20), being therefore not visible or apparent to the user. The steps of the training workflow (200) may result in a set of AI models that are introduced in the pre-operative diagnosis of the patient's problem, corresponding to the steps of automatic segmentation and classification (2101), automatic landmarks detection (2102), model for automatic classification of bone density (2103), and model for automatic osteophytes detection (2104).

To be able to perform the pre-operative analysis, medical images stored in a database may undergo acquisition and study (201). The images used for the training include but are not limited to:
Portable Network Graphics (*.png);
Joint Photographic Group image (*.jpg);
Tagged Image File Format (*tiff);
Digital Radiography (X-ray);
Computed Tomography (CT);
Magnetic Resonance Imaging (MRI); or
Positron Emission Tomography (PET).

A manual labelling procedure (202) may occur. The manual labelling procedure (202) may include a person that manually analyzes and studies each medical image. In some embodiments, the person may include a professional prepared and/or accredited for the analysis and study of medical images. The person may, upon the analysis and study, identify and manually label landmarks and bones continuously to create an annotated datasets.

The annotated datasets may then be sent through a script to AI training models (203) to acquire the detection and classification of landmarks and bones knowledge based on the annotated ground truth of the dataset.

The method for bone segmentation and classification and landmarks detection may include one or more image processing, computer vision and/or machine learning techniques. Computer vision is a scientific field that describes the process of a machine understanding images and videos and is being used in orthopedics to aid in diagnostic decision-making. Likewise, Machine Learning (ML) has been successfully used in different orthopedic applications, most commonly for automatic assessment of plain film radiographs in various applications. ML algorithms are commonly used to analyze medical images and segment bones for surgery using image recognition tools. Following this training, the AI models are capable of automatically detecting and classifying bones and landmarks (204).

Following the AI training model (203) to detect and classify bones and landmarks, the overall software application (20) may use a DevOps-enabled infrastructure and process to evolve and improve the software at a faster pace in comparison to the traditional software development and infrastructure management processes. Further, the method for automatic bone segmentation and classification and landmarks detection (204) relies on image processing, computer vision and machine learning and by performing the training the system can generate AI models (205) that are able to automatically detect and classify bones and landmarks more quickly and efficiently.

The next step, described as bone quality evaluation (206), includes two parallel procedures: the model classification for bone density (2061) and the model for osteophytes detection (2062).

Bone density, or Bone Mineral Density (BMD) refers to the amount of bone mineral in bone tissue and is measured to indicate if a patient has osteoporosis, osteopenia or if there is a high fracture risk. This information is relevant for the pre-operative diagnosis since it has an impact on the implant selection, as well as the implant's size. In this step, a model is therefore developed to automatically detect bone density (2061) and to adjust the pre-operative planning according to the patient's mineral density.

On the other hand, osteophytes are bone spurs that grow on the bones of the spine or around the joints. The integration of this model allows the detection of the presence of (2062) and this information will be included in the pre-operative diagnosis (210).

The output results are then checked and measured in terms of accuracy (207), resorting to the use of technologies based on microservices architecture that allow software development to focus on building single-function modules with well-defined interfaces and operations. To perform this feature, Machine Learning, AI based algorithms, Computer vision, Computer Graphics, Medical Imaging, Data Processing and Deep Learning technologies are used. The result accuracy testing (207) is therefore performed simultaneously through an automatic testing (2071), where the models are continually tested and refined by the system to achieve the best results quickly and accurately, and through human verification (2072), where the models are manually verified to assess if the pre-operative diagnosis was performed in accordance with high standards requirements for its performance.

Following the result accuracy testing (207), the provided results are assessed if they meet the specifications (208) and fulfills the intended purpose and are in accordance with quality control. If the results are not satisfactory (2081), the models are sent back to the AI models training to detect and classify bones and landmarks (204). If the results are satisfactory (2082), then the models are included in the system (209) through the pre-operative diagnosis step (210). The pre-operative diagnosis (210) is composed of a couple of procedures, which include automatic segmentation and classification (2101), automatic landmarks detection (2102), model for automatic classification of bone density (2103) and model for automatic osteophytes detection (2104).

At this stage, automatic segmentation and classification (2101), and the equivalent model has been tested and verified. In some embodiments, based on the test and verification, the software application (20) may automatically perform automatic segmentation and classification (2101) based on the AI learning performed in previous steps. The automatic segmentation and classification (2101) step make use of Deep Learning technology to detect the bones that are important to the procedure, as well as to accurately classify them. It also uses an algorithm that allows the extraction of a polygonal mesh of an isosurface from a three-dimensional scalar field, called voxels.

Upon completion of testing and verification of the automatic landmarks detection (2102), the software application (20) may perform the automatic landmarks detection (2102) based on the AI learning performed in previous steps. The automatic landmark detection (2102) step makes use of Deep Learning technology to detect the relevant landmarks for the procedure, as well as to accurately classify them. It also uses an algorithm that allows the extraction of a polygonal mesh of an isosurface from a three-dimensional scalar field, called voxels.

Based on the model for bone density detection and the AI training (2061), the system is able to automatically perform the automatic classification of the bone's mineral density (2103). This automatic classification of bone density (2103) step makes use of Deep Learning technology to detect the landmarks important to the procedure, as well as to accurately classify them.

Based on the model for osteophytes detection and the AI training, the system becomes able to automatically perform the automatic classification of the osteophytes (2104). This automatic osteophyte detection (2104) step makes use of Deep Learning technology to detect the landmarks important to the procedure, as well as to accurately classify them.

When completed, the pre-operative diagnosis (210) resulting from the training workflow (200) is sent to the pre-operative workflow (100), in particular to the bone model and landmark position generation (103). The pre-operative diagnosis (210) is presented in the software application (20) user interface (11), where the user is able to visualize the plan and interact with it-zoom, rotation, etc.

It is understood that at least one aspect/functionality of various embodiments described herein can be performed in real-time and/or dynamically. As used herein, the term “real-time” is directed to an event/action that can occur instantaneously or almost instantaneously in time when another event/action has occurred. For example, the “real-time processing,” “real-time computation,” and “real-time execution” all pertain to the performance of a computation during the actual time that the related physical process (e.g., a user interacting with an application on a mobile device) occurs, in order that results of the computation can be used in guiding the physical process.

As used herein, the term “dynamically” and term “automatically,” and their logical and/or linguistic relatives and/or derivatives, mean that certain events and/or actions can be triggered and/or occur without any human intervention. In some embodiments, events and/or actions in accordance with the present disclosure can be in real-time and/or based on a predetermined periodicity of at least one of: nanosecond, several nanoseconds, millisecond, several milliseconds, second, several seconds, minute, several minutes, hourly, several hours, daily, several days, weekly, monthly, etc.

In some embodiments, exemplary inventive, specially programmed computing systems and platforms with associated devices are configured to operate in the distributed network environment, communicating with one another over one or more suitable data communication networks (e.g., the Internet, satellite, etc.) and utilizing one or more suitable data communication protocols/modes such as, without limitation, IPX/SPX, X.25, AX.25, AppleTalk™, TCP/IP (e.g., HTTP), near-field wireless communication (NFC), RFID, Narrow Band Internet of Things (NBIOT), 3G, 4G, 5G, GSM, GPRS, WiFi, WiMax, CDMA, satellite, ZigBee, and other suitable communication modes.

The material disclosed herein may be implemented in software or firmware or a combination of them or as instructions stored on a machine-readable medium, which may be read and executed by one or more processors. A machine-readable medium may include any medium and/or mechanism for storing or transmitting information in a form readable by a machine (e.g., a computing device). For example, a machine-readable medium may include read only memory (ROM); random access memory (RAM); magnetic disk storage media; optical storage media; flash memory devices; electrical, optical, acoustical, or other forms of propagated signals (e.g., carrier waves, infrared signals, digital signals, etc.), and others.

Computer-related systems, computer systems, and systems, as used herein, include any combination of hardware and software. Examples of software may include software components, programs, applications, operating system software, middleware, firmware, software modules, routines, subroutines, functions, methods, procedures, software interfaces, application program interfaces (API), instruction sets, computer code, computer code segments, words, values, symbols, or any combination thereof. Determining whether an embodiment is implemented using hardware elements and/or software elements may vary in accordance with any number of factors, such as desired computational rate, power levels, heat tolerances, processing cycle budget, input data rates, output data rates, memory resources, data bus speeds and other design or performance constraints.

One or more aspects of at least one embodiment may be implemented by representative instructions stored on a machine-readable medium which represents various logic within the processor, which when read by a machine causes the machine to fabricate logic to perform the techniques described herein. Such representations, known as “IP cores” may be stored on a tangible, machine readable medium and supplied to various customers or manufacturing facilities to load into the fabrication machines that make the logic or processor. Of note, various embodiments described herein may, of course, be implemented using any appropriate hardware and/or computing software languages (e.g., C++, Objective-C, Swift, Java, JavaScript, Python, Perl, QT, etc.).

In some embodiments, one or more of illustrative computer-based systems or platforms of the present disclosure may include or be incorporated, partially or entirely into at least one personal computer (PC), laptop computer, ultra-laptop computer, tablet, touch pad, portable computer, handheld computer, palmtop computer, personal digital assistant (PDA), cellular telephone, combination cellular telephone/PDA, television, smart device (e.g., smart phone, smart tablet or smart television), mobile internet device (MID), messaging device, data communication device, and so forth.

As used herein, term “server” should be understood to refer to a service point which provides processing, database, and communication facilities. By way of example, and not limitation, the term “server” can refer to a single, physical processor with associated communications and data storage and database facilities, or it can refer to a networked or clustered complex of processors and associated network and storage devices, as well as operating software and one or more database systems and application software that support the services provided by the server. Cloud servers are examples.

In some embodiments, as detailed herein, one or more of the computer-based systems of the present disclosure may obtain, manipulate, transfer, store, transform, generate, and/or output any digital object and/or data unit (e.g., from inside and/or outside of a particular application) that can be in any suitable form such as, without limitation, a file, a contact, a task, an email, a message, a map, an entire application (e.g., a calculator), data points, and other suitable data. In some embodiments, as detailed herein, one or more of the computer-based systems of the present disclosure may be implemented across one or more of various computer platforms such as, but not limited to: (1) FreeBSD, NetBSD, OpenBSD; (2) Linux; (3) Microsoft Windows™; (4) OpenVMS™; (5) OS X (MacOS™); (6) UNIX™; (7) Android; (8) iOS™; (9) Embedded Linux; (10) Tizen™; (11) WebOS™; (12) Adobe AIR™; (13) Binary Runtime Environment for Wireless (BREW™); (14) Cocoa™ (API); (15) Cocoa™ Touch; (16) Java™ Platforms; (17) JavaFX™; (18) QNX™; (19) Mono; (20) Google Blink; (21) Apple WebKit; (22) Mozilla Gecko™; (23) Mozilla XUL; (24). NET Framework; (25) Silverlight™; (26) Open Web Platform; (27) Oracle Database; (28) Qt™; (29) SAP NetWeaver™; (30) Smartface™; (31) Vexi™; (32) Kubernetes™ and (33) Windows Runtime (WinRT™) or other suitable computer platforms or any combination thereof. In some embodiments, illustrative computer-based systems or platforms of the present disclosure may be configured to utilize hardwired circuitry that may be used in place of or in combination with software instructions to implement features consistent with principles of the disclosure. Thus, implementations consistent with principles of the disclosure are not limited to any specific combination of hardware circuitry and software. For example, various embodiments may be embodied in many different ways as a software component such as, without limitation, a stand-alone software package, a combination of software packages, or it may be a software package incorporated as a “tool” in a larger software product.

For example, exemplary software specifically programmed in accordance with one or more principles of the present disclosure may be downloadable from a network, for example, a website, as a stand-alone product or as an add-in package for installation in an existing software application. For example, exemplary software specifically programmed in accordance with one or more principles of the present disclosure may also be available as a client-server software application, or as a web-enabled software application. For example, exemplary software specifically programmed in accordance with one or more principles of the present disclosure may also be embodied as a software package installed on a hardware device.

In some embodiments, illustrative computer-based systems or platforms of the present disclosure may be configured to handle numerous concurrent users that may be, but is not limited to, at least 100 (e.g., but not limited to, 100-999), at least 1,000 (e.g., but not limited to, 1,000-9,999), at least 10,000 (e.g., but not limited to, 10,000-99,999), at least 100,000 (e.g., but not limited to, 100,000-999,999), at least 1,000,000 (e.g., but not limited to, 1,000,000-9,999,999), at least 10,000,000 (e.g., but not limited to, 10,000,000-99,999,999), at least 100,000,000 (e.g., but not limited to, 100,000,000-999,999,999), at least 1,000,000,000 (e.g., but not limited to, 1,000,000,000-999,999,999,999), and so on.

In some embodiments, illustrative computer-based systems or platforms of the present disclosure may be configured to output to distinct, specifically programmed graphical user interface implementations of the present disclosure (e.g., a desktop, a web app., etc.). In various implementations of the present disclosure, a final output may be displayed on a displaying screen which may be, without limitation, a screen of computer, a screen of a mobile device, or the like. In various implementations, the display may be a holographic display. In various implementations, the display may be a transparent surface that may receive a visual projection. Such projections may convey various forms of information, images, or objects. For example, such projections may be a visual overlay for a mobile augmented reality (MAR) application.

As used herein, terms “cloud,” “Internet cloud,” “cloud computing,” “cloud architecture,” and similar terms correspond to at least one of the following: (1) a large number of computers connected through a real-time communication network (e.g., Internet); (2) providing the ability to run a program or application on many connected computers (e.g., physical machines, virtual machines (VMs)) at the same time; (3) network-based services, which appear to be provided by real server hardware, and are in fact served up by virtual hardware (e.g., virtual servers), simulated by software running on one or more real machines (e.g., allowing to be moved around and scaled up (or down) on the fly without affecting the end user).

In some embodiments, the illustrative computer-based systems or platforms of the present disclosure may be configured to securely store and/or transmit data by utilizing one or more of encryption techniques (e.g., private/public key pair, Triple Data Encryption Standard (3DES), block cipher algorithms (e.g., IDEA, RC2, RC5, CAST and Skipjack), cryptographic hash algorithms (e.g., MD5, RIPEMD-160, RTR0, SHA-1, SHA-2, Tiger (TTH), WHIRLPOOL, RNGS).

As used herein, the term “user” shall have a meaning of at least one user. In some embodiments, the terms “user”, “subscriber” “consumer” or “customer” should be understood to refer to a user of an application or applications as described herein and/or a consumer of data supplied by a data provider. By way of example, and not limitation, the terms “user” or “subscriber” can refer to a person who receives data provided by the data or service provider over the Internet in a browser session, or can refer to an automated software application which receives the data and stores or processes the data.

The aforementioned examples are, of course, illustrative and not restrictive.

Publications cited throughout this document are hereby incorporated by reference in their entirety. While one or more embodiments of the present disclosure have been described, it is understood that these embodiments are illustrative only, and not restrictive, and that many modifications may become apparent to those of ordinary skill in the art, including that various embodiments of the inventive methodologies, the illustrative systems and platforms, and the illustrative devices described herein, can be utilized in any combination with each other. Further still, the various steps may be carried out in any desired order (and any desired steps may be added and/or any desired steps may be eliminated).

1. A computer-implemented method for orthopedic surgery planning, the method comprising the steps of:
importing at least one orthopedic medical image of a patient into a software application;
selecting a medical procedure to which to apply the imported orthopedic medical image;
generating a bone model and a landmark position from the imported orthopedic medical image, wherein the bone model and the landmark position comprise information from a pre-operative diagnosis, the pre-operative diagnosis comprising at least automatic bone segmentation and classification,
automatic landmark detection,
automatic classification of bone density, and
automatic osteophytes detection;
adjusting the landmark position generated from the imported orthopedic medical image;
automatically creating a pre-operative planning proposal;
validating the proposed pre-operative planning proposal; and
exporting an orthopedic surgery planning data file.

2. Computer-implemented method according to claim 1, wherein the generated bone model allows at least one of visualizing, rotating, zooming, interacting by a user, and adjusting the landmark position to refine the landmark position.

3. Computer-implemented method according to claim 1, wherein the automatic pre-operative planning proposal comprises automatic bone alignment based on clinical angles, automatic bone resections, automatic template dimensioning and placement procedures, and user preferences.

4. Computer implemented method according to claim 1, wherein the automatic pre-operative planning proposal comprises at least one of a landmark and/or template repositioning; measurement of distances and/or angles; intersecting template 3D models and anatomical structure 3D models; anatomical structure 3D models resecting; template 3D models dimensioning or replacement; or zooming, in a 3D environment, allowing the manual adjustment and refinement of the automatic pre-operative planning proposal.

5. Computer-implemented method according to claim 3, wherein the user preferences comprise a pre-operative planning user adjustment, a user preference setting, a manual review of user preferences for integration within the training workflow, an AI training module, and a user preferences statistical model.

6. Computer-implemented method according to claim 3, wherein the user preferences comprise a manual review of user preferences to train the AI models based on annotated datasets.

7. Computer-implemented method according to claim 5, wherein the pre-operative planning user adjustment comprises validation of the automatic pre-operative planning proposal.

8. Computer-implemented method according to claim 1, wherein the orthopedic surgery planning data file comprises at least one of an exporting, downloading, saving in a document format, printing, sending to a PACS, exporting 3D bone models, exporting 3D template information comprising implant brand, implant size, anatomical position, amount of bone resection, and integrating with external devices or software for the purposes of surgical execution.

9. A computer implemented training workflow method, the method comprising:
acquiring and storing orthopedic medical images;
labeling the stored orthopedic medical images in annotated datasets;
providing the labeled datasets to AI models for training the AI models;
generating, based on the training, trained AI models comprising i) trained AI models for detecting and classifying bones and landmarks and ii) trained AI models for bone quality evaluation;
detecting and classifying bones and landmarks in orthopedic medical images through the trained AI models for detecting and classifying bones and landmarks;
performing an evaluation of bone quality using the trained AI models for bone quality evaluation;
performing accuracy testing of the trained AI Models;
analyzing results of the accuracy testing;
sending the results to re-train the AI models for detecting and classifying bones and landmarks in case of a negative analysis of the results; and
providing including the trained AI models to perform the pre-operative diagnosis in case of a positive analysis of the results.

10. Computer-implemented method according to any claim 9, wherein labeling stored orthopedic medical images in annotated datasets comprises labeling anonymized imported medical images of a patient and the acquired and stored orthopedical medical images.

11. Computer-implemented method according to any of claim 9, wherein the AI models are trained based on the annotated datasets to detect and classify bones and landmarks, to perform the pre-operative diagnosis.

12. Computer-implemented method according to claim 9, wherein the trained AI models for bone quality evaluation of comprising a bone density classification model and a model for osteophytes detection, and wherein the AI models for bone quality evaluation are used to perform the pre-operative diagnosis.

13. Computer-implemented method according to claim 9, wherein the accuracy testing comprises the steps of automatic testing and human verification, and, in case of a positive analysis of the results, the results are included in the system to generate the pre-operative diagnosis.

14. Computer-implemented method according to claim 9, wherein detecting and classifying bones and landmarks comprise adjusting landmark position by the user of the imported orthopedic medical image or rejecting result analysis of the accuracy testing.

15. (canceled)

16. (canceled)

17. The method of claim 9 further comprising:
performing the pre-operative diagnosis for bone model and landmark position generation.
